(19) United States
(12) Patent Application Publication
(10) Pub. No.: US 2022/0287950 A1
(43) Pub. Date: Sep. 15, 2022

(54) MICROPARTICLE-STRUCTURED THICKENER HAVING IMPROVED SENSATION OF USE

(71) Applicant: LG HOUSEHOLD & HEALTH CARE LTD., Seoul (KR)

(72) Inventors: Hyo Jin BONG, Seoul (KR); Jung Eun BAE, Seoul (KR); Woo Sun SHIM, Seoul (KR)

(73) Assignee: LG HOUSEHOLD & HEALTH CARE LTD., Seoul (KR)

(21) Appl. No.: 17/754,112

(22) PCT Filed: Sep. 24, 2020
(86) PCT No.: PCT/KR2020/012951

§ 371 Date: Mar. 24, 2022

(30) Foreign Application Priority Data
Sep. 24, 2019 (KR) 10-2019-0117774
Sep. 10, 2020 (KR) 10-2020-0116124

(51) Int. Cl. A61K 8/81; A61Q 19/00; A61K 8/06

(52) U.S. Cl. CPC A61K 8/8188; A61Q 19/00; A61K 8/062; A61K 2800/48

(57) ABSTRACT

The present invention relates to a microparticle-structured thickener having improved sensation of use. Even when swelling in an aqueous phase, the thickener according to the present invention shows the advantage of retaining the form of particles as well as appearing transparent and being high in stability because the particles disperse homogeneously.

### TECHNICAL FIELD

The present invention relates to a microparticle-structured thickener having an improved feeling of use.

### BACKGROUND ART

Conventional water-dispersible thickeners have the disadvantages of having spreadability with softness and a feeling of thickness, a poor feeling of finish, and high stickiness as a larger amount thereof is used.

Accordingly, 2-acrylamido-2-methylpropane sulfonic acid (AMPS)-based thickeners having a silky feeling of use have been reported. The AMPS-based thickeners are generally known to be synthesized by precipitation polymerization (Patent Document 1). Since the particles of thickeners synthesized by the precipitation polymerization are present in an amorphous state, they are dispersed and swell in an irregular form in an aqueous phase, thereby increasing the viscosity of a formulation and imparting stability. However, the thickeners obtained by the precipitation polymerization are opaque when dispersed in an aqueous phase, which makes it difficult to apply the thickener to a transparent formulation. Therefore, there is a concern of limitations in developing various formulations.

### RELATED-ART DOCUMENTS

### Patent Documents

1. EP Patent Publication No. 1746114

### DISCLOSURE

### Technical Problem

The present invention is directed to providing a microparticle-structured thickener having an improved feeling of use.

### Technical Solution

One aspect of the present invention provides a method of preparing a thickener, which includes: preparing an oil-in-water emulsion composition including: an aqueous phase including a compound represented by the following Chemical Formula 1 and a crosslinkable monomer; and an oil phase including a non-polar organic solvent having a solubility parameter of 15 (MPa)^{1/2} or more; preparing a water-in-oil inverse emulsion composition by heating to 60° C. or more; and preparing a cross-linked polymer by adding a reaction initiator and performing a polymerization reaction.

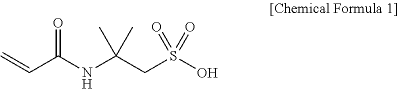

Another aspect of the present invention provides a composition including a thickener prepared by the above-described method.

### Advantageous Effects

In the present invention, a homogeneous microparticle-type thickener can be prepared by allowing a polymerization reaction to proceed in a uniform inverse emulsion phase having a small particle size using a phase inversion temperature (PIT) polymerization method. The spherical particle-type thickener prepared according to the present invention can have an advantage of not only maintaining the form of particles even when swelling in an aqueous phase but also having a transparent appearance and high stability due to dispersion of uniform particles.

Most of the thickeners synthesized by precipitation polymerization are dispersed in an aqueous phase while having a tangled chain structure in an amorphous form, whereas the thickener prepared (polymerized) according to the present invention is dispersed in the form of microparticles in water and thus can maintain its form even in a formulation. Also, when the thickener according to the present invention is applied to the skin, the physical bonding structure between the particles is easily broken, and thus a feeling of use such as spreadability, moisturizing, nourishing, and the like can be improved.

### DESCRIPTION OF DRAWINGS

FIG. 1 shows an image illustrating the comparison of the transparencies of thickeners prepared in some examples of the present invention.

FIGS. 2 and 4 show the results of measuring the form of spherical microparticles prepared in some examples of the present invention.

FIG. 3 shows images illustrating the comparison of the particles of thickeners prepared in Example 2 and Comparative Example 1 of the present invention.

FIG. 5 shows the results of measuring the rheological properties of thickeners prepared in some examples of the present invention.

FIG. 6 shows optical images of creams prepared in some examples of the present invention.

### MODES OF THE INVENTION

The present invention provides a method of preparing a thickener, which includes the steps of:

(1) preparing an oil-in-water emulsion composition including: an aqueous phase including a compound represented by the following Chemical Formula 1 and a crosslinkable monomer; and an oil phase including a non-polar organic solvent having a solubility parameter of 15 (MPa)^{1/2} or more;

(2) preparing a water-in-oil inverse emulsion composition by heating to 60° C. or more; and

(3) preparing a cross-linked polymer by adding a reaction initiator and performing a polymerization reaction.

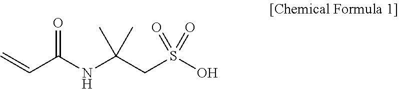

Hereinafter, the configuration of the present invention will be described in detail.

In the present invention, a thickener is prepared using a phase inversion temperature (PIT) polymerization method.

Specifically, in the present invention, a PIT polymerization method, in which an oil-in-water (o/w) emulsion phase formed at room temperature based on an appropriate combination of a non-polar organic solvent having a solubility parameter of 15 (MPa)^{1/2} or more, water, and a surfactant (specifically, two types of surfactants, a surfactant having an HLB value of 3 to 8 and a surfactant having an HLB value of 8 to 16, or a different two types of surfactants, a surfactant having an HLB value of 5 to 7 and a surfactant having an HLB value of 9 to 11) is converted into a water-in-oil (w/o) emulsion phase at or above a specific temperature, is used to allow a polymerization reaction to proceed in a uniform inverse emulsion phase having small particle sizes. Through the above method, in the present invention, a homogeneous microparticle-type thickener can be prepared. The spherical microparticle-type thickener polymerized by the above method has an advantage of not only maintaining the form of particles even when swelling in an aqueous phase but also having a transparent appearance and high stability due to dispersion of uniform particles.

In the present invention, a “thickener” means a cross-linked polymer prepared by the preparation method of the present invention and may mean a solution in which the cross-linked polymer is dispersed.

In the present invention, the step (1) is a step of preparing an oil-in-water emulsion composition including: an aqueous phase including a compound represented by Chemical Formula 1 and a crosslinkable monomer; and an oil phase including a non-polar organic solvent having a solubility parameter of 15 (MPa)^{1/2} or more.

The aqueous phase includes a compound represented by Chemical Formula 1 and a crosslinkable monomer.

In an embodiment, the compound represented by Chemical Formula 1 may be 2-acrylamido-2-methylpropane sulfonic acid (AMPS).

Although there is no particular limitation on the amount of the compound represented by Chemical Formula 1, the amount may be 95 to 99.5 parts by weight with respect to the total weight (100 parts by weight) of the compound represented by Chemical Formula 1 and the crosslinkable monomer.

In an embodiment, the crosslinkable monomer may be a compound having two or more acrylate groups, two or more acrylamide groups, or two or more vinyl groups. As the crosslinkable monomer, for example, one or more selected from the group consisting of trimethylolpropane ethoxylate triacrylate (TMPETA), methylenebisacrylamide, divinyl sulfone, divinyl benzene, divinyl ether, divinyl acetylene, polyglycol diacrylate, polyglycol triacrylate, 4-arm-polyglycol tetraacrylate, and the like may be used. In the present invention, specifically, TMPETA, which is a compound represented by the following Chemical Formula 2, may be used. The TMPETA is a compound having three crosslinking points and is capable of enhancing the molecular weight of a cross-linked product and accordingly enhancing viscosity.

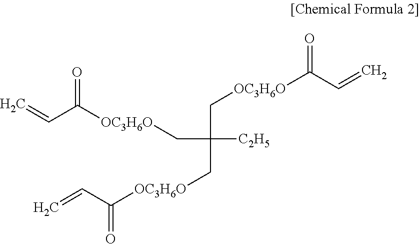

Although there is no particular limitation on the amount of the crosslinkable monomer, the amount may be 0.5 to 5.0 parts by weight with respect to the total weight (100 parts by weight) of the compound represented by Chemical Formula 1 and the crosslinkable monomer.

In an embodiment, a solvent of the aqueous phase may be water (distilled water).

In an embodiment, the pH of the aqueous phase may be 5 to 9. The pH may be adjusted using ammonia water.

In the present invention, the oil phase includes an organic solvent and a surfactant.

In an embodiment, the organic solvent may be a non-polar organic solvent having a solubility parameter of 15 (MPa)^{1/2} or more, specifically, a hydrocarbon oil having 7 to 17 carbon atoms or a straight-chain saturated hydrocarbon oil having 7 to 17 carbon atoms. The solubility parameter is well known to those skilled in the art and is customarily used as a guideline for determining the compatibility and solubility of a substance.

In the present invention, heptane may be used as the non-polar organic solvent.

Heptane has a boiling point of 98° C. and is not restricted in the polymerization process according to the present invention as compared to other organic solvents. Hexane, which is one of the organic solvents, has a boiling point of 68° C., and thus it is not possible to raise a synthesis temperature to 60° C. or more, and therefore, there are many restrictions on the process. Also, during the process, there is a risk that the temperature inside a reactor may increase by about 5 to 10° C. due to the occurrence of heat generation resulting from the explosive reaction during an initiator input process. A method of slowly inputting an initiator to maintain the homeostasis of the reaction temperature causes an increase in process time when applied to a mass production process, thereby leading to an increase in production costs. Also, when the temperature increases above the boiling point, the reactant is subjected to bumping such that the risk of explosion of the reactor increases, and therefore, it is important to manage the heat of reaction in mass production. In the present invention, a desired thickener of the present invention can be prepared without any restrictions on the process by using heptane as the organic solvent.

In an embodiment, as the surfactant, one or more surfactants may be used. The HLB value of the surfactant may be 6 to 14, 7 to 10, or 8 to 9. In this case, a total HLB value may mean the HLB value of one surfactant when one surfactant is used and the sum of the HLB values of two or more surfactants when two or more surfactants are used. When the total HLB value is 6 or less, there is a water-in-oil inverse emulsion at room temperature due to the strong hydrophobicity of the surfactant, and thus polymerization proceeds without an inversion process, thereby the effect of reducing the size of the emulsion may be insufficient. On the other hand, when the total HLB value is 14 or more, an oil-in-water emulsion whose appearance is in an aqueous phase is maintained due to the strong hydrophilicity of the surfactant, and thus spherical particles are not formed, resulting in concerns that the amorphous state of the prepared thickener may increase.

In an embodiment, as the surfactant, two types of surfactants may be used. In this case, a combination of a surfactant having an HLB value of 3 to 8 and a surfactant having an HLB value of 8 to 16 may be used, or a combination of a surfactant having an HLB value of 5 to 7 and a surfactant having an HLB value of 9 to 11 may be used.

In the present invention, a combination of polyoxyethylene (3) oleyl ether (HLB=6.6) and polyoxyethylene (6) oleyl ether (HLB=9.6) may be used as the surfactant. Specifically, polyoxyethylene (3) oleyl ether (HLB=6.6) and polyoxyethylene (6) oleyl ether (HLB=9.6) may be used in combination in a weight ratio of 1:1. In this case, an HLB value may be 8.1.

Although there is no particular limitation on the amount of the surfactant, the amount may vary depending on the type of organic solvent. Specifically, the amount of the surfactant may be 5 to 30 parts by weight with respect to the total weight (100 parts by weight) of the organic solvent. Within the above-described content range, inversion of an emulsion is possible, and PIT polymerization can be easily performed.

In an embodiment, a weight ratio of the aqueous phase and the oil phase may be 30:70 to 70:30.

In the present invention, the step (2) is a step of preparing a water-in-oil inverse emulsion composition by heating to 60° C. or more.

The emulsion prepared in the step (1) is in an oil-in-water form at room temperature and may be inverted into a water-in-oil emulsion by heating. The emulsion produced by the inversion may have the effect of reducing the size of the emulsion and also has the effect that a polymer prepared in a step to be described below has a small particle size.

In an embodiment, a reaction temperature may specifically be 62° C. or more, 65° C. or more, 70° C. or more, or 75° C., and the upper limit thereof may be 100° C. Within the above-described temperature range, a thickener can be stably prepared.

In the present invention, the step (3) is a step of preparing a cross-linked polymer by adding a reaction initiator and performing a polymerization reaction.

In an embodiment, the type of reaction initiator is not particularly limited as long as it has an ability to initiate radical polymerization and may be selected from the group consisting of a peroxide and an azo compound. As the peroxide initiator, benzoyl peroxide, acetyl peroxide, dilauryl peroxide, di-tert-butyl peroxide, cumyl hydroperoxide, hydrogen peroxide, potassium persulfate, or the like may be used, and as the azo compound initiator, azo nitrile, azo ester, azo amide, azo imidazoline, azo amidine, Macro azo initiator, or the like may be used. In the present invention, 4,4′-azobis(4-cyanovaleric acid) may be used as the initiator.

In the present invention, after the preparation of the inverse emulsion, an initiator may be added, and reaction may be performed to obtain a cross-linked polymer, that is, a thickener, having a desired viscosity of the present invention, and the yield of the thickener may be increased. In the present invention, a solution containing the cross-linked polymer prepared by the above-described method may be used as a thickener, and the solution may be precipitated with a precipitating agent to finally prepare a powder-type thickener. In this case, acetone or the like may be used as the precipitating agent.

In an embodiment, this step may be performed at 65° C. or more for 2 to 6 hours.

The present invention also provides a thickener prepared by the above-described thickener preparation method.

The thickener according to the present invention is in the form of spherical microparticles and swells in water, ethanol, or a water-ethanol liquid mixture, thereby providing a visually-uniform highly-viscous solution.

The thickener prepared according to the present invention may have an average particle diameter of 10 to 300 um.

In addition, the thickener prepared according to the present invention may have a viscosity of 500 to 70,000 cps or 5,000 to 50,000 cps as measured in a 1% (w/v) aqueous dispersion at 25° C.

The present invention also provides a cosmetic composition including the above-described thickener.

The amount of the thickener is not particularly limited and may be appropriately determined depending on a desired cosmetic. In terms of usability, the thickener may be included in an amount of 0.01 to 10 wt % or 0.1 to 5 wt %.

According to a formulation, the cosmetic may be prepared by appropriately mixing with oily bases, surfactants, powders, moisturizers, UV absorbers, alcohols, chelating agents, pH adjusters, preservatives, antioxidants, thickeners, drugs, dyes, pigments, fragrances, water, or the like within a range that does not impair the effects of the present invention.

The type of cosmetic according to the present invention is not particularly limited. For example, the cosmetic may be applied to a basic cosmetic, a makeup cosmetic, a pack cosmetic, a hair cosmetic, and the like.

In addition, the thickener according to the present invention may be used in a medicine, a food stabilizer, or a lubricant composition. In this case, the amount of the thickener may be adjusted appropriately depending on an intended purpose, and known additional ingredients may be included.

Hereinafter, the present invention will be described in detail through examples. However, the following examples are merely presented to exemplify the present invention, and the scope of the present invention is not limited to the following examples. That is, the examples of the present invention serve to complete the disclosure of the present invention and are provided to fully inform the scope of the invention to those of ordinary knowledge and skill in the art to which this invention pertains. This invention should be defined based on the scope of the appended claims.

### EXAMPLES

### Comparative Example 1

17 g of 2-acrylamido-2-methylpropane sulfonic acid (AMPS) and 6.5 g of ammonium bicarbonate were added to and sufficiently dissolved in 100 g of tert-butanol as a solvent at 70° C., and then 1.6 g of trimethylolpropane triacrylate as a crosslinking agent was dissolved in 6.4 g of tert-butanol and added. Finally, 0.2 g of dilauroyl peroxide was added and allowed to react for 2 hours, and the resultant was precipitated with acetone to obtain a thickener.

### Example 1

Aqueous phase: 17 g of AMPS and 0.5 g of trimethylolpropane ethoxylate triacrylate (TMPETA) were dissolved in 63 g of distilled water, and the resultant was neutralized to pH 7 to 9 using ammonia water.

Oil phase: 10 g of each of polyoxyethylene (3) oleyl ether and polyoxyethylene (6) oleyl ether were added to and mixed well with 80 g of heptane.

The aqueous phase and the oil phase were input into a reactor and heated to 70° C., then 0.1 g of 4,4′-azobis(4-cyanovaleric acid) as an initiator was added and allowed to react for 3 hours, and the resultant was precipitated with acetone to obtain a thickener.

### Example 2

Aqueous phase: 17 g of AMPS and 1.0 g of TMPETA were dissolved in 63 g of distilled water, and the resultant was neutralized to pH 7 to 9 using ammonia water.

Oil phase: 10 g of each of polyoxyethylene (3) oleyl ether and polyoxyethylene (6) oleyl ether were added to and mixed well with 80 g of heptane.

The aqueous phase and the oil phase were input into a reactor and heated to 70° C., then 0.1 g of 4,4′-azobis(4-cyanovaleric acid) as an initiator was added and allowed to react for 3 hours, and the resultant was precipitated with acetone to obtain a thickener.

### Example 3

Aqueous phase: 17 g of AMPS and 1.5 g of TMPETA were dissolved in 63 g of distilled water, and the resultant was neutralized to pH 7 to 9 using ammonia water.

Oil phase: 10 g of each of polyoxyethylene (3) oleyl ether and polyoxyethylene (6) oleyl ether were added to and mixed well with 80 g of heptane.

The aqueous phase and the oil phase were input into a reactor and heated to 70° C., then 0.1 g of 4,4′-azobis(4-cyanovaleric acid) as an initiator was added and allowed to react for 3 hours, and the resultant was precipitated with acetone to obtain a thickener.

### Comparative Example 2

Aqueous phase: 17 g of AMPS and 1.0 g of TMPETA were dissolved in 63 g of distilled water, and the resultant was neutralized to pH 7 to 9 using ammonia water.

Oil phase: 10 g of polyoxyethylene (3) oleyl ether and 5 g of polyoxyethylene (6) oleyl ether were added to and mixed well with 80 g of hexane.

The aqueous phase and the oil phase were input into a reactor and heated to 70° C., then 0.1 g of 4,4′-azobis(4-cyanovaleric acid) as an initiator was added and allowed to react for 3 hours, and the resultant was precipitated with acetone to obtain a thickener.

### Experimental Example 1. Comparison of Transparency

The transparencies of the compositions (thickeners) prepared in Comparative Example 1 and Example 2 were compared.

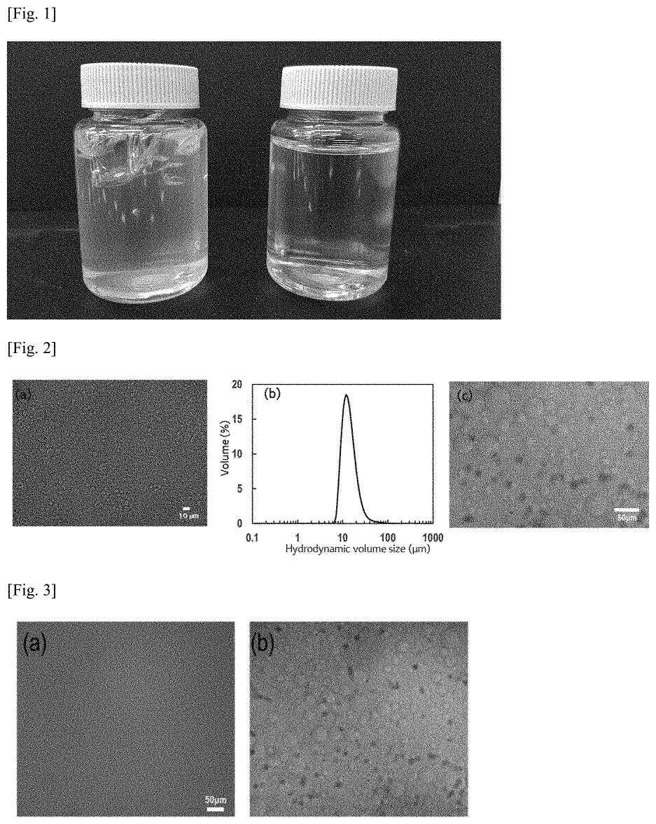

A result thereof is shown in FIG. 1.

In FIG. 1, the left container contains the 1% (w/v) aqueous solution of the thickener polymerized in Comparative Example 1, and the right container contains the 1% (w/v) aqueous solution of the thickener polymerized in Example 2.

As shown in FIG. 1, it can be confirmed that the thickener prepared in Comparative Example 1 was opaque, whereas the thickener prepared in Example 2 was transparent in the aqueous solution.

### Experimental Example 2. Measurement of Spherical Particle Form

The form of spherical particles prepared in the examples and comparative examples was measured.

FIG. 2 shows particles of the thickener prepared in Example 2. Specifically, FIG. 2A shows the droplet state (70° C.) of the oil-in-water emulsion formed in the polymerization reaction in Example 2, FIG. 2B shows the hydrodynamic volume size the polymerized microparticle-type thickener, and FIG. 2C shows an optical image of the thickener-containing aqueous solution.

As shown in FIG. 2, it can be confirmed that the thickener prepared in Example 2 was present in the form of spherical particles.

FIG. 3 shows results (optical images) illustrating the comparison of the particles of the thickeners prepared in Comparative Example 1 (a) and Example 2 (b).

As shown in FIG. 3, microparticles could not be confirmed in the thickener polymerized in Comparative Example 1. On the other hand, it can be confirmed that the thickener polymerized in Example 2 was present in the form of microparticles in the aqueous solution.

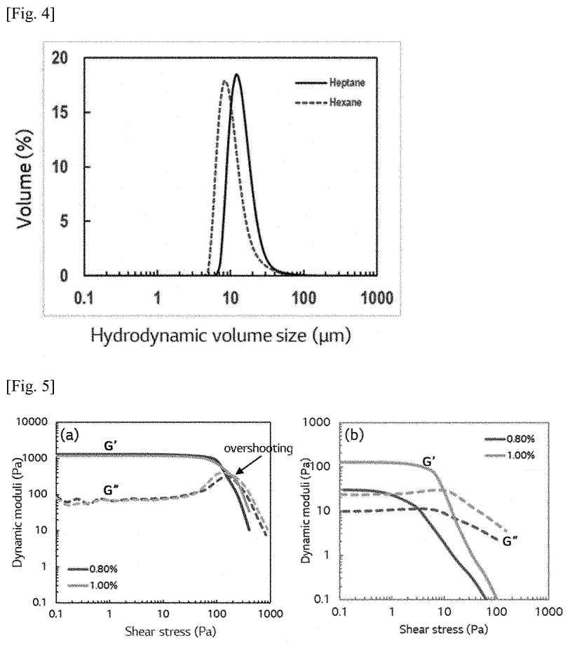

Meanwhile, FIG. 4 shows results illustrating the comparison of the hydrodynamic volume size of the microparticle-type thickeners polymerized in Example 2 and Comparative Example 2.

As shown in FIG. 4, it can be confirmed that the thickener of Example 2 using heptane as an organic solvent had a larger volume size than that when hexane was used.

### Experimental Example 3. Comparison of Viscosity

The viscosity of the thickeners prepared in Example 2 and Comparative Example 2 was measured under conditions of a 64 spindle and 10 to 30 rpm using Brookfield LVT (Brookfield, Mass.).

The viscosity was measured for an aqueous solution containing the thickener at 1% (w/v).

As a result, the viscosity of the thickener-containing aqueous solution of Comparative Example 2 was 2,000 cps, and the viscosity of the thickener-containing aqueous solution of Example 2 was 14,000 cps.

From the measurement results, it can be confirmed that the viscosity of the thickener according to the present invention was improved compared to that of Comparative Example.

### Experimental Example 4. Comparison of Rheological Properties

The rheological properties of the prepared thickener were measured. The rheological properties were measured using a Discovery hr-3 hybrid rheometer (TA Instruments).

Results thereof are shown in FIG. 5.

FIGS. 5A and 5B show results of measuring the rheological properties of the thickeners prepared in Comparative Example 1 and Example 2, respectively.

Rheologically, the polymer chains in a polymer solution have a tangled structure, and the tangled structure is loosened at high shear stress. Just before the structure is completely loosened, a phenomenon in which the loosening of the structure is resisted occurs. This is referred to as an overshooting phenomenon, and it can be confirmed that the phenomenon occurred in the case of Comparative Example 1 in an amorphous state.

On the other hand, it can be confirmed that since the thickener of Example 2 was present in the form of microparticles, the overshooting phenomenon hardly occurred at high shear stress, and thus the structure collapsed. Due to this rheological feature, the thickener according to the present invention is considered to have an improved feeling of use, especially, improved spreadability, in a cosmetic formulation.

### Preparation Example 1. Emulsion Cream Formulation

Creams were prepared with the ingredients and contents shown in the following Table 1.

TABLE 1

| | | Cream 1 | Cream 2 | Cream 3 | Cream 4 | Cream 5 |
|---|---|---|---|---|---|---|
| Oil phase | Silicone oil | 10.0 | 10.0 | 10.0 | 10.0 | 10.0 |
| | Glycerin | 8.0 | 8.0 | 8.0 | 8.0 | 8.0 |
| | Heptandiol | 8.0 | 8.0 | 8.0 | 8.0 | 8.0 |
| | Hexadiol | 1.0 | 1.0 | 1.0 | 1.0 | 1.0 |
| | Hydrogenated | 0.5 | 0.5 | 0.5 | 0.5 | 0.5 |
| | castor oil | | | | | |
| Aqueous | Comparative | 0.4 | — | — | — | |
| phase | Example 1 | | | | | |
| | Example 1 | — | 0.4 | — | — | |
| | Example 2 | — | — | 0.4 | — | |
| | Example 3 | — | — | — | 0.4 | |
| | Comparative | — | — | — | — | 0.4 |
| | Example 2 | | | | | |
| | Dexpanthenol | 1.0 | 1.0 | 1.0 | 1.0 | 1.0 |
| | Trisodium | 0.02 | 0.02 | 0.02 | 0.02 | 0.02 |
| | EDTA | | | | | |
| | Distilled water | To 100 | To 100 | To 100 | To 100 | To 100 |

### Experimental Example 4. Evaluation of Feeling of Use by Panelists

(1) Method

Twenty trained panelists were selected to evaluate a feeling of use.

The evaluation of a feeling of use was performed based on a 5-point scale with respect to softness, a feeling of thickness, moisturizing, and stickiness.

(2) Result

Results thereof are shown in the following Table 2.

TABLE 2

| | Softness | Feeling of thickness | Moisturizing | Stickiness |
|---|---|---|---|---|
| Cream 1 | 3.2 | 3.5 | 3.7 | 1.3 |
| Cream 2 | 3.2 | 2.8 | 3.8 | 1.2 |
| Cream 3 | 4.1 | 3.7 | 3.7 | 1.0 |
| Cream 4 | 3.0 | 3.4 | 3.6 | 1.2 |
| Cream 5 | 3.0 | 1.5 | 4.2 | 1.0 |

As shown in Table 2, it can be confirmed that the cream 3 received the highest evaluation in terms of softness and a feeling of thickness.

Specifically, it can be confirmed that the viscosity of the 0.4% (w/v) aqueous solution of the thickener of Comparative Example 1 was 5,500 cps, which decreased to 4,000 cps when applied to cream 1, whereas the viscosity of the 0.4% (w/v) aqueous solution of the thickener of Example 2 was 1,500 cps, which increased to 6,500 cps when applied to cream 3.

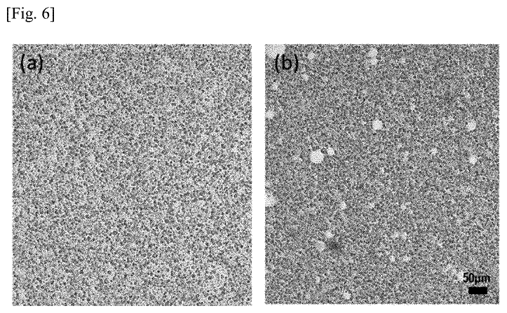

Meanwhile, FIG. 6 show optical images of cream 1 (a) and cream 3 (b).

As shown in FIG. 6, as a result of examining the formulations using an optical microscope, it can be confirmed that the spherical particles of the thickener coexisted in the formulation, which affected the fluidity of the formulation to form a more stable formulation.

The thickener of Comparative Example 1 had decreased viscosity due to the contraction of the tangled structure caused by an emulsifying agent in the formulation, whereas the particle-type thickener of Example 2 had a hydrodynamic volume and thus maintained a certain volume in the formulation without being significantly affected by an emulsifying agent, and the coexistence of emulsion particles and microparticles, which affects the fluidity in the formulation, is considered to double the thickening effect.

### INDUSTRIAL APPLICABILITY

In the present invention, a homogeneous microparticle-type thickener can be prepared by allowing a polymerization reaction to proceed in a uniform inverse emulsion phase having a small particle size using a phase inversion temperature (PIT) polymerization method. The spherical particle-type thickener prepared according to the present invention can have an advantage of not only maintaining the form of particles even when swelling in an aqueous phase but also having a transparent appearance and high stability due to dispersion of uniform particles.

1. A method of preparing a thickener, comprising:
preparing an oil-in-water emulsion composition including: an aqueous phase including a compound represented by the following Chemical Formula 1 and a crosslinkable monomer; and an oil phase including a non-polar organic solvent having a solubility parameter of 15 (MPa)^{1/2} or more;
preparing a water-in-oil inverse emulsion composition by heating to 60° C. or more; and
preparing a cross-linked polymer by adding a reaction initiator and performing a polymerization reaction:

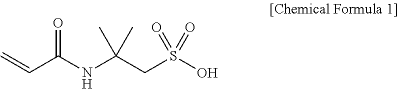

2. The method of claim 1, wherein the compound represented by Chemical Formula 1 is 2-acrylamido-2-methylpropane sulfonic acid (AMPS).

3. The method of claim 1, wherein an amount of the compound represented by Chemical Formula 1 is 95 to 99.5 parts by weight with respect to the total weight of the compound represented by Chemical Formula 1 and the crosslinkable monomer.

4. The method of claim 1, wherein the crosslinkable monomer is a compound having two or more acrylate groups, two or more acrylamide groups, or two or more vinyl groups.

5. The method of claim 1, wherein an amount of the crosslinkable monomer is 0.5 to 5 parts by weight with respect to the total weight of the compound represented by Chemical Formula 1 and the crosslinkable monomer.

6. The method of claim 1, wherein the non-polar organic solvent is a hydrocarbon oil having 7 to 17 carbon atoms.

7. The method of claim 1, wherein the oil phase further includes one or more surfactants, and
the surfactant has a total HLB value of 6 to 14.

8. The method of claim 7, wherein two types of surfactants, a surfactant having an HLB value of 3 to 8 and a surfactant having an HLB value of 8 to 16, are used.

9. The method of claim 1, wherein the reaction initiator is one or more selected from the group consisting of a peroxide and an azo compound.

10. The method of claim 1, wherein the polymerization reaction is performed at 60° C. or more for 2 to 6 hours.

11. A composition comprising a thickener prepared by the method according to claim 1.

12. The composition of claim 11, which is for preparation of a cosmetic, a medicine, a food stabilizer, or a lubricant.
